(19) United States
(12) Patent Application Publication
(10) Pub. No.: US 2025/0303074 A1
(43) Pub. Date: Oct. 2, 2025

(54) NEEDLESTICK PREVENTION DEVICES

(71) Applicant: SUZHOU HEALTHY TREE MEDICAL TECHNOLOGY CO., LTD., Suzhou (CN)

(72) Inventors: Changyan XIONG, Suzhou (CN); Xingwu LI, Suzhou (CN)

(73) Assignee: SUZHOU HEALTHY TREE MEDICAL TECHNOLOGY CO., LTD., Suzhou (CN)

(21) Appl. No.: 18/621,232

(22) Filed: Mar. 29, 2024

(51) Int. Cl. A61M 5/32

(52) U.S. Cl. CPC A61M 5/3245; A61M 5/3275; A61M 2005/3268

(57) ABSTRACT

Disclosed a needlestick prevention device, comprising an injector component, an accumulation member, a fixing sleeve, and a limiting sleeve. The injector component includes a pressing cap, an injector syringe flanging, an injector syringe, and a needle. The fixing sleeve includes a locking clip, a flanging fixing clip, at least one support plate, and a limiting plate. The limiting sleeve includes at least one guiding feature, at least one first limiting hole, at least one second limiting hole, and a limiting buckle. In a first state, the accumulation member is accumulated, and the fixing sleeve is in a first position. In a second state, the locking clip rotates to separate from the limiting buckle, the fixing sleeve is released from the limiting sleeve, and the accumulation member is released. In a third state, the accumulation member is completely released, and the fixing sleeve and the injector component move along the at least one guiding feature to a second position.

### TECHNICAL FIELD

The present disclosure relates to the technical field of injection devices, and in particular to a needlestick prevention device.

### BACKGROUND

Needlestick injuries are prone to occur when injection devices are used in a medical setting. Needlestick injuries refer to the risk of puncture wounds or cross-infection caused by a user unintentionally sticking a needle into himself or another person before or after using an injection device. To reduce the needlestick injuries, the relevant authorities impose stricter regulations on medical instruments, requiring that disposable injectors should be equipped with specialized protective devices. Conventional protective devices are complex in structure, costly, and complicated to operate, affecting the operating efficiency.

Therefore, it is desirable to provide a needlestick prevention device which ensures that a needle can be safely covered or retracted during and after an injection process, further improving the safety and ease of use of an injector.

### SUMMARY

One or more embodiments of the present disclosure provide a needlestick prevention device. The needlestick prevention device may comprise an injector component, an accumulation member, a fixing sleeve, and a limiting sleeve. The fixing sleeve and the accumulation member may be arranged in the limiting sleeve. One end of the accumulation member may be fixedly connected with the fixing sleeve, and another end of the accumulation member may be fixedly connected with the limiting sleeve. The injector component may include a pressing cap, an injector syringe flanging, an injector syringe, and a needle. The fixing sleeve may include one or more locking clips, a flanging fixing clip, at least one support plate, and a limiting plate. The flange fixing clip may be configured to fix the injector syringe flanging. The limiting sleeve may include at least one guiding feature, at least one first limiting hole, at least one second limiting hole, and a limiting buckle. The needlestick prevention device may have a first state, a second state, and a third state. In the first state, the accumulation member may be accumulated, and the fixing sleeve may be in a first position. When the fixing sleeve is in the first position, the limiting buckle is connected with the locking clips, the at least one support plate may be in the at least one second limiting hole, and the limiting plate may be at one end of the at least one guiding feature near the at least one second limiting hole. In the second state, the locking clips may rotate to separate from the limiting buckle, the fixing sleeve may be released from the limiting sleeve, and the accumulation member may be released. In the third state, the accumulation member may be released completely, and the fixing sleeve and the injector component may move along the at least one guiding feature to the second position. When the fixing sleeve is in the second position, the at least one support plate may be in the at least one first limiting hole, and the limiting plate may be at one end of the at least one guiding feature near the at least one first limiting hole.

### BRIEF DESCRIPTION OF THE DRAWINGS

The present disclosure will be further illustrated by way of exemplary embodiments, which will be described in detail with reference to the accompanying drawings. These embodiments are not limiting, and in these embodiments, the same numbering denotes the same structure, wherein:

FIG. 1 is a schematic diagram illustrating an exemplary overall structure of a needlestick prevention device according to some embodiments of the present disclosure;

FIG. 2 is a schematic structural diagram illustrating an exemplary cross section of an injector component according to some embodiments of the present disclosure;

FIG. 3 is a schematic diagram illustrating an exemplary stereoscopic structure of a fixing sleeve according to some embodiments of the present disclosure;

FIG. 4 is a schematic structural diagram illustrating an exemplary cross section of a limiting sleeve according to some embodiments of the present disclosure;

FIG. 5 is a schematic structural diagram illustrating an exemplary needlestick prevention device in a first state according to some embodiments of the present disclosure;

FIG. 6 is a schematic structural diagram illustrating an exemplary needlestick prevention device in a second state according to some embodiments of the present disclosure;

FIG. 7 is a schematic structural diagram illustrating an exemplary needlestick prevention device in a third state according to some embodiments of the present disclosure;

FIG. 8 is a schematic structural diagram illustrating an exemplary cross section of a fixing sleeve according to some embodiments of the present disclosure;

FIG. 9 is a schematic structural diagram illustrating an exemplary cross section of a pressing cap according to some embodiments of the present disclosure;

FIG. 10 is a schematic diagram illustrating an exemplary connecting strip according to some embodiments of the present disclosure;

FIG. 11 is a schematic structural diagram illustrating an exemplary cross section of an accumulation member when the accumulation member is accumulated according to some embodiments of the present disclosure;

FIG. 12 is a schematic structural diagram illustrating an exemplary cross section of an accumulation member when the accumulation member is released according to some embodiments of the present disclosure;

FIG. 13 is a schematic structural diagram illustrating an exemplary cross section of an accumulation member when the accumulation member is a spring according to some embodiments of the present disclosure;

FIG. 14 is schematic diagram illustrating an exemplary local structure of a fixing sleeve according to some embodiments of the present disclosure;

FIG. 15 is schematic diagram illustrating an exemplary local structure of a limiting sleeve according to some embodiments of the present disclosure;

FIG. 16 is a schematic diagram illustrating an exemplary local structure of at least one support plate and a limiting plate according to some embodiments of the present disclosure;

FIG. 17 is a schematic diagram illustrating an exemplary unfolding of an inner wall structure of a limiting sleeve according to some embodiments of the present disclosure;

FIG. 18 is a top view illustrating an exemplary second limiting hole according to some embodiments of the present disclosure;

FIG. 19 is a top view illustrating an exemplary guiding feature according to some embodiments of the present disclosure;

FIG. 20 is a schematic diagram illustrating an exemplary adjustment portion according to some embodiments of the present disclosure;

FIG. 21 is a schematic structural diagram illustrating an exemplary needlestick prevention device in a first state according to some embodiments of the present disclosure;

FIG. 22 is a schematic diagram illustrating an exemplary local enlargement of a needlestick prevention device in a first state according to some embodiments of the present disclosure;

FIG. 23 is a schematic diagram illustrating an exemplary release member of a needlestick prevention device according to some embodiments of the present disclosure;

FIG. 24 is a schematic diagram illustrating an exemplary local structure of a needlestick prevention device according to some embodiments of the present disclosure;

FIG. 25 is a cross-sectional diagram illustrating an exemplary fixing protrusion and a fixing cavity according to some embodiments of the present disclosure;

FIG. 26 is a stereoscopic diagram illustrating an exemplary fixing protrusion and a fixing cavity according to some embodiments of the present disclosure;

FIG. 27 is a schematic diagram illustrating an exemplary position of an adjustment member according to some embodiments of the present disclosure;

FIG. 28 is a schematic diagram illustrating an exemplary position of an adjustment member according to some embodiments of the present disclosure; and

FIG. 29 is a schematic diagram illustrating an exemplary adjustment member according to some embodiments of the present disclosure.

### DETAILED DESCRIPTION

Exemplary embodiments or implementations will be described herein in detail, examples of which are represented in the accompanying drawings. When the following description relates to the accompanying drawings, the same numerals in the different accompanying drawings denote the same or similar elements unless otherwise indicated. The embodiments described in the following examples are not intended to be representative of all embodiments consistent with the present disclosure. Rather, they are only examples of apparatuses and methods that are consistent with some aspects of the present disclosure as detailed in the appended claims.

Terms used in the present disclosure are used solely for the purpose of describing particular embodiments and are not intended to limit the present disclosure. As used in the present disclosure and the appended claims, the singular forms of “a”, “the” and “said” are also intended to include the plural form, unless the context clearly indicates otherwise.

It should be understood that the terms “first,” “second,” or the like as used in the specification of the present disclosure and the claims do not indicate any order or importance, but are merely used to distinguish between different components. Similarly, the words “one”, “a”, or the like, do not indicate a quantitative limitation, but rather the presence of at least one component. Unless otherwise noted, “anterior”, “posterior”, “lower”, and/or “upper,” and similar terms are used for illustrative purposes only and are not limited to a location or a spatial orientation. The words “including”, “comprising”, or the like, are intended to refer to the elements or objects that appear before “including” or “comprising” may include or comprise the elements or objects listed after “including” or “comprising” and equivalents thereof, and do not exclude other elements or objects.

Arranging a specialized protective device for a disposable injector can reduce needlestick injuries by guaranteeing that a needle can be safely covered or retracted after an injection process is completed. In existing scenarios, the injector equipped with the protective device is mainly an “active” injector that requires a user to perform a certain specific action to activate the protective device. Therefore, in the existing healthcare scenarios, after the injection process is completed, a healthcare worker needs to manually push the needle into the protective device (e.g., a needle shield) to cover the used needle for recovery. This manual retraction of the needle is also one of the causes of needlestick injuries. In addition, existing protective devices are often designed as the needle shields, which are also not capable of meeting higher safety needs.

Accordingly, one or more embodiments of the present disclosure provide a needlestick prevention device, which may, by release of an accumulation member and mating of a fixing sleeve and a limiting sleeve at different phases of the injection process, quickly retract an injection needle, thereby reducing the risk of needlestick injuries during and after the injection process. The needlestick prevention device may also fix and limit an injector component through one or more locking clips, a limiting buckle, or the like, thereby ensuring the safety of the device in different states.

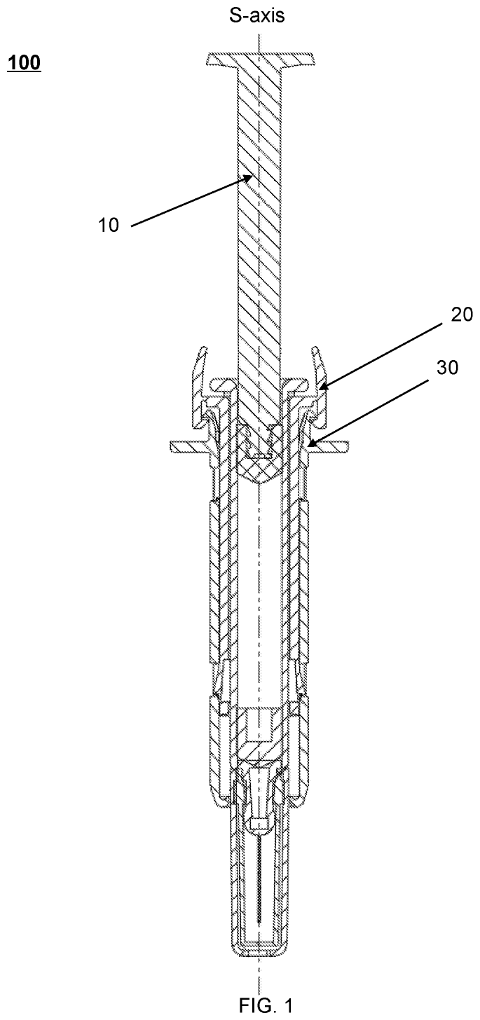

FIG. 1 is a schematic diagram illustrating an exemplary overall structure of a needlestick prevention devices according to some embodiments of the present disclosure.

In some embodiments, as illustrated in FIG. 1, a needlestick prevention device 100 may include an injector component 10, an accumulation member, a fixing sleeve 20, and a limiting sleeve 30. The fixing sleeve 20 and the accumulation member may be arranged in the limiting sleeve 30. One end of the accumulation member may be fixedly connected with the fixing sleeve 20, and another end of the accumulation member may be fixedly connected with the limiting sleeve 30. In some embodiments, the limiting sleeve 30 may sleeve outside the fixing sleeve 20. The limiting sleeve 30 and the fixing sleeve 20 may be coaxially arranged. The limiting sleeve 30 and the fixing sleeve 20 may slide relatively along a central axis.

The central axis refers to a common axis of the limiting sleeve 30 and the fixing sleeve 20. For example, as illustrated in FIG. 1, the central axis may be an S-axis. The injector component 10 may perform an injection along an S-axis direction. The limiting sleeve 30 and the fixing sleeve 20 may slide relatively along the S-axis direction.

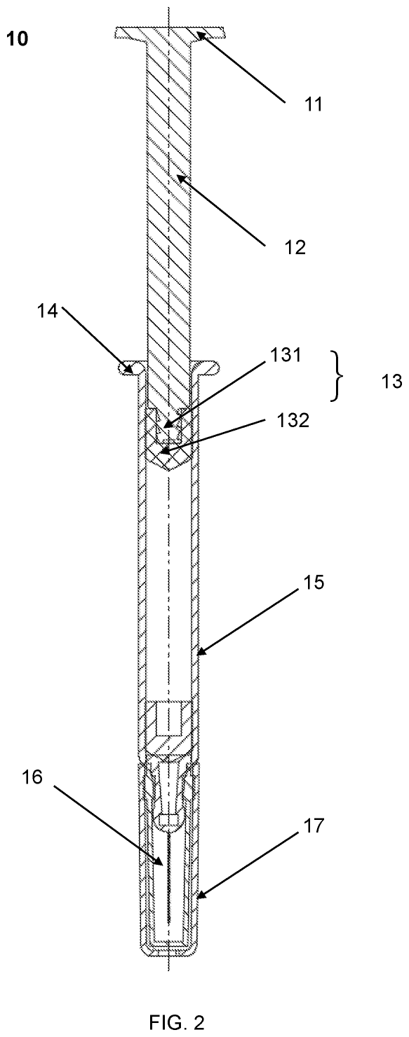

FIG. 2 is a schematic structural diagram illustrating an exemplary cross section of an injector component according to some embodiments of the present disclosure.

The injector component 10 refers to a combination of parts and/or structures for completing an injection. In some embodiments, as illustrated in FIG. 1, the injector component 10 and the fixing sleeve 20 may be coaxially arranged.

In some embodiments, as illustrated in FIG. 2, the injector component 10 may include a pressing cap 11, an injector syringe flanging 14, an injector syringe 15, and a needle 16.

The pressing cap 11 refers to a part for receiving a push force. By applying the push force to the pressing cap 11, the injector component 10 may be pressed downward to complete the injection.

In some embodiments, the pressing cap 11 may be arranged as a predetermined shape structure, such as a circle, an ellipse, or another structure with a rounded edge, to avoid scratching the user. In some embodiments, an upper surface of the pressing cap 11 may be a flat or curved plane to facilitate pressing. In some embodiments, the injector component 10 may further include a rod 12. The rod 12 refers to a component for transmitting the push force. In some embodiments, a cross-sectional area of the pressing cap 11 may be greater than a cross-sectional area of the rod 12, i.e., the pressing cap 11 may be provided as a cap structure, thereby facilitating the application of the push force to the rod 12.

The injector syringe flanging 14 refers to a protruding structure for fixing the injector syringe 15 to the fixing sleeve 20. In some embodiments, the injector syringe flanging 14 may be provided at a top of the injector syringe 15. The injector syringe flanging 14 may relatively fix the injector syringe 15 and the fixing sleeve 20.

The injector syringe 15 may be configured to store an injection liquid. The injector syringe 15 may be provided as a hollow structure. The needle 16 may be configured to implement the injection of the injection liquid. The needle 16 may be provided at a lower end of the injector syringe 15 and fixedly connected with the injector syringe 15.

In some embodiments, the injector component 10 may include a plunger 13. The plunger 13 may include a rubber plug 131 and a push plug 132. The rubber plug 131 and the push plug 132 may be removably fixed. A pre-filled medication may be provided between the push plug 132 and the needle 16. The pre-filled medication refers to a medicament pre-filled in the injector syringe.

In some embodiments, as illustrated in FIG. 2, the plunger 13 may be provided at a lower end of the rod 12. The rubber plug 131 may be provided at an upper end of the plunger 13. The push plug 132 may be provided at a lower end of the plunger 13.

In some embodiments, the injector component may further include a needle cap 17.

During an assembly phase, the needle cap 17 may wrap and be fixed to an outer side of the needle 16. Before the injection, the needle cap 17 may be removed for the injection, and the rod 12 may be manually pushed forward to complete the injection of the pre-filled medication. In some embodiments, after the injection is completed, the needle cap 17 may be refixed to the outer side of the needle 16 to form protection, or the needle cap 17 may be directly discarded, and the needle 16 may retract into the limiting sleeve 30 along with retraction of the fixing sleeve 20.

In some embodiments, before the injection, the needle cap may form the protection for the needle to prevent external germs from contaminating the pre-filled medication through the needle; and further to reduce personnel injuries caused by needle exposure.

The accumulation member refers to a part configured to provide a resilience force to the fixing sleeve 20. In some embodiments, the accumulation member may be configured as an elastic structure. The resilience force may be provided to the fixing sleeve 20 by compressing or stretching the elastic structure. More descriptions regarding the accumulation member may be found in the description below.

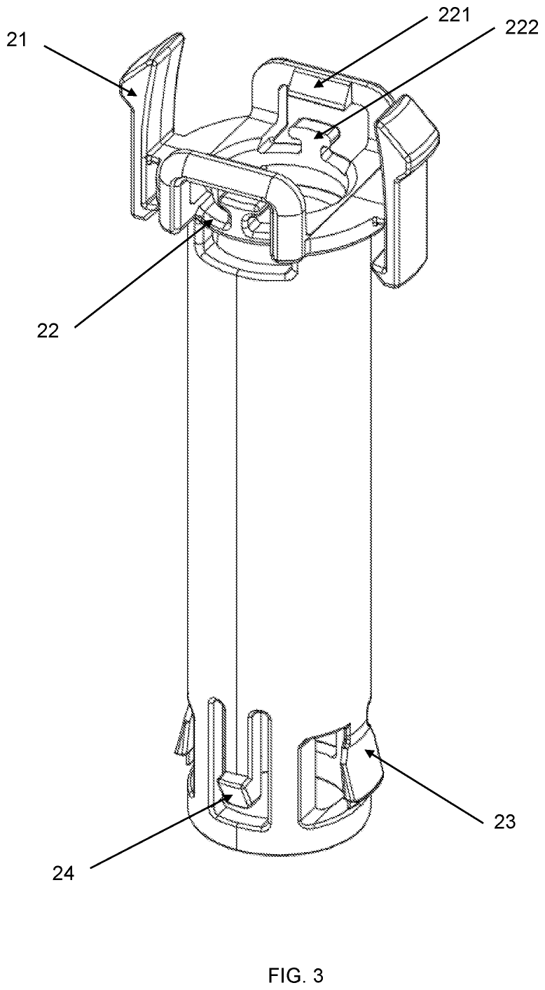

FIG. 3 is a schematic diagram illustrating an exemplary stereoscopic structure of a fixing sleeve according to some embodiments of the present disclosure.

The fixing sleeve 20 refers to a part for fixing the injector syringe 15. The fixing sleeve 20 may be a hollow structure that sleeves outside the injector syringe 15. In some embodiments, as illustrated in FIG. 3, the fixing sleeve 20 may include one or more locking clips 21, a flanging fixing clip 22, at least one support plate 23, and a limiting plate 24.

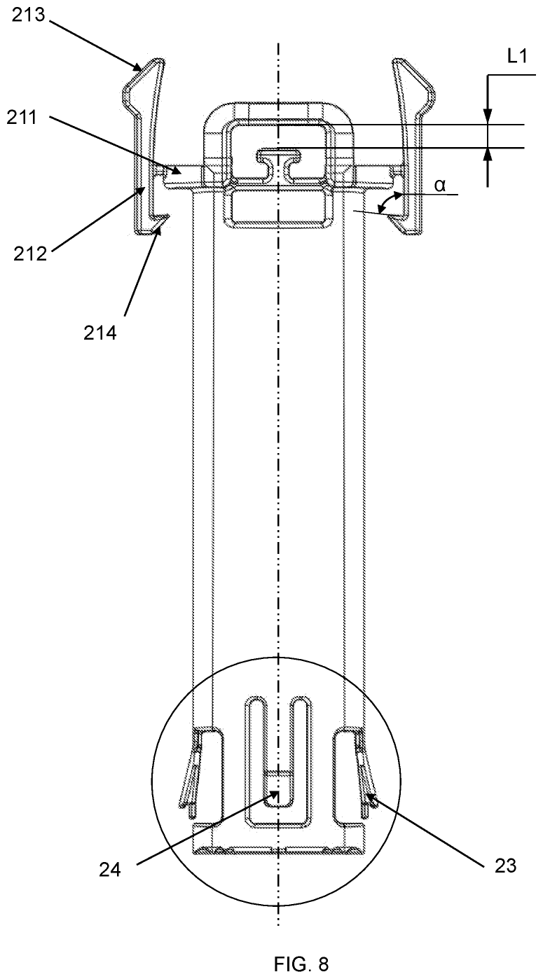

The locking clips 21 refer to parts for locking the fixing sleeve 20 to the limiting sleeve 30. In some embodiments, as illustrated in FIG. 3, the locking clips 21 may be provided at an outer side of an upper end of the fixing sleeve 20. More descriptions regarding the locking clips 21 may be found in the related descriptions of FIG. 8.

The flanging fixing clip 22 may be configured to fix the injector syringe flanging 14. The fixing sleeve 20 may be fixedly connected with the injector syringe flanging 14 through the flanging fixing clip 22. In some embodiments, as illustrated in FIG. 3, the flanging fixing clip 22 may be provided at an end of the fixing sleeve 20.

The at least one support plate 23 refers to a structure for fixing the fixing sleeve 20 to the limiting sleeve 30. In some embodiments, in a natural state, the at least one support plate 23 may be an outward open structure, as illustrated in FIG. 3; when subjected to an external force, the at least one support plate 23 may contract inward. In some embodiments, as illustrated in FIG. 3, the at least one support plate 23 may be provided at a lower end of the fixing sleeve 20.

The limiting plate 24 refers to a protruding structure provided on a surface of the fixing sleeve 20 for limiting a relative rotation of the fixing sleeve 20 from the limiting sleeve 30. In some embodiments, the limiting plate 24 may be provided on a surface of the lower end of the fixing sleeve 20.

In some embodiments, the limiting plate 24 may be spaced apart from the at least one support plate 23.

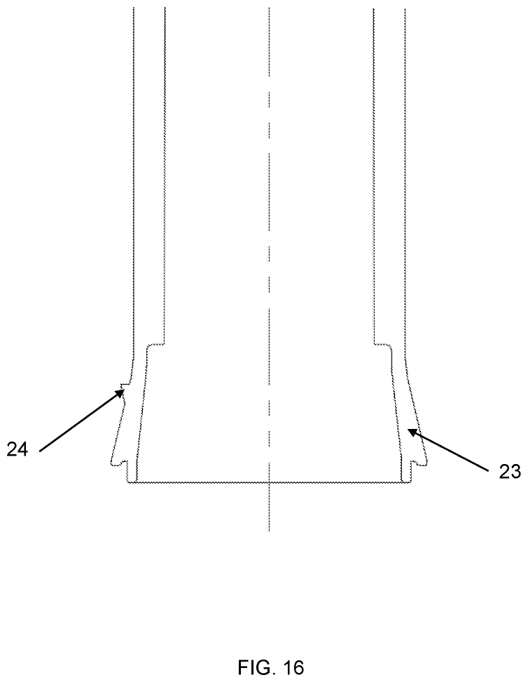

In some embodiments, the limiting plate 24 may be disposed on the at least one support plate 23. More descriptions may be found in FIG. 16 and related descriptions thereof.

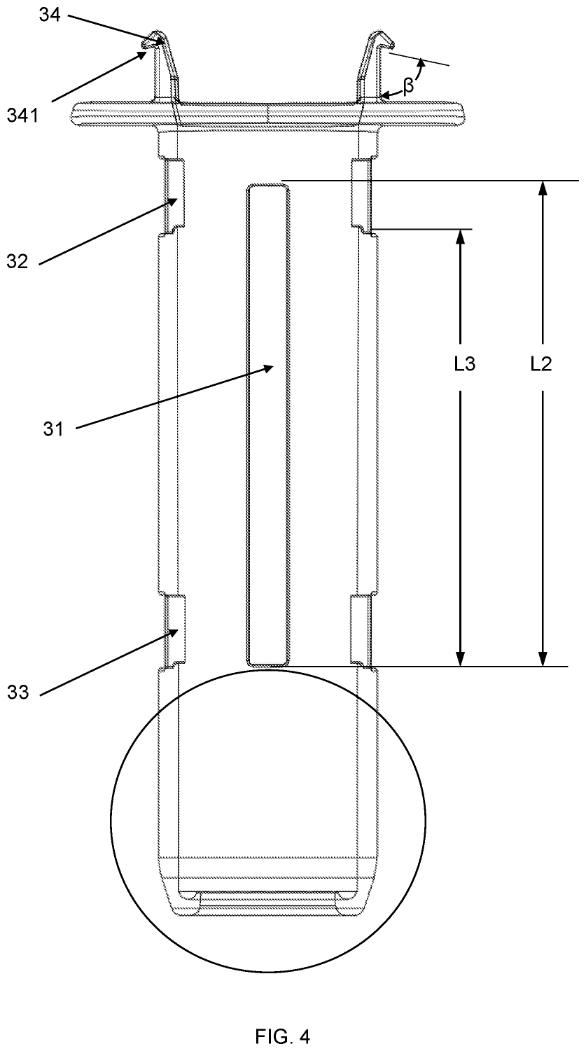

FIG. 4 is a schematic structural diagram illustrating an exemplary cross section of a limiting sleeve according to some embodiments of the present disclosure.

The limiting sleeve 30 refers to a part for limiting a position of the fixing sleeve 20. In some embodiments, as illustrated in FIG. 4, the limiting sleeve 30 may include at least one guiding feature 31, at least one first limiting hole 32, at least one second limiting hole 33, and a limiting buckle 34.

The at least one guiding feature 31 refers to a structure for guiding a movement of the fixing sleeve 20. The at least one guiding feature 31 may be located in a middle of the limiting sleeve 30. The at least one guiding feature 31 may mate with the limiting plate 24 to guide the fixing sleeve 20 relatively slide with the limiting sleeve 30 along a direction of a central axis, and may also limit the fixing sleeve 20 from relatively rotating with the limiting sleeve 30.

The at least one first limiting hole 32 and the at least one second limiting hole 33 may be configured to limit a relative position of the fixing sleeve and the limiting sleeve. In some embodiments, as illustrated in FIG. 4, the at least one first limiting hole 32 and the at least one second limiting hole 33 may be provided in an outer upper portion and an outer lower portion, respectively, of the fixing sleeve 20. The at least one first limiting hole 32 and the at least one second limiting hole 33 may be oppositely arranged along the central axis. A count of the at least one first limiting hole 32 may be the same as a count of the at least one second limiting hole 33.

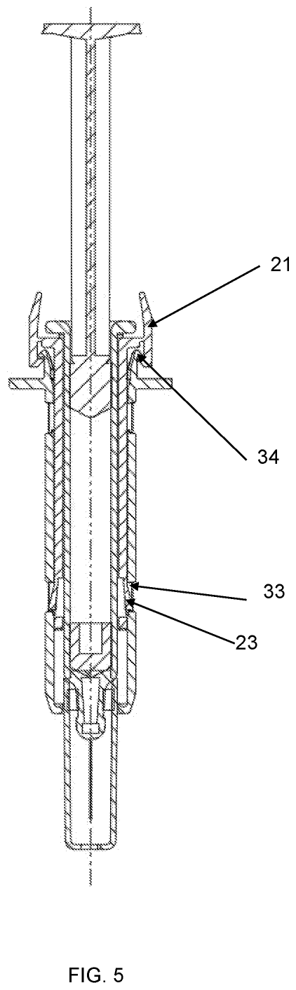

The limiting buckle 34 refers to a buckle structure for fixing the fixing sleeve 20. In some embodiments, as illustrated in FIG. 5, the limiting buckle 34 may be arranged at an upper end of the limiting sleeve 30. The limiting buckle 34 and the locking clips 21 may mate to realize a fixed connection between the limiting sleeve 30 and the fixing sleeve 20.

In some embodiments, the at least one guiding feature 31 may be a groove. In some embodiments, a length of the groove may be greater than a travel of the at least one support plate 23 moving from the at least one second limiting hole 33 to the at least one first limiting hole 32. Merely by way of example, as illustrated in FIG. 4, a length L2 of the at least one guiding feature 31 may be greater than a travel L3 of the at least one support plate 23 moving from the at least one second limiting hole 33 to the at least one first limiting hole 32.

In some embodiments, a width of the groove may match a width of the at least one support plate 23.

In some embodiments, the at least one guiding feature may mate with the at least one support plate, so that the problem that the fixing sleeve deviates during sliding, causing the at least one support plate to not match the at least one first limiting hole or the at least one second limiting hole can be avoided, thereby improving the limiting accuracy of the needlestick prevention device. Meanwhile, the length of the at least one guiding feature may be greater than the travel of the at least one support plate moving from the at least one second limiting hole to the at least one first limiting hole, so that when the resilience force applied to the fixing sleeve by the accumulation member is too large and the at least one support plate slides out of the at least one first limiting hole, the at least one guiding feature may limit the limiting plate from continuing to move, thereby limiting the fixing sleeve from continuing to move. In this way, a limiting protection may be formed for the fixing sleeve to prevent the fixing sleeve from slipping out.

In some embodiments, the limiting sleeve 30 may further include a protection plate which may be provided on an outer side of the limiting buckle 34. The protection plate may include various structures, such as a U-shaped structure, etc.

In some embodiments, the protection plate may be configured to protect the limiting buckle or the locking clips, avoiding accidental contact with the locking clips during operation.

More descriptions regarding the limiting sleeve 30 may be found in the following description.

In some embodiments, the needlestick prevention device 100 may have a first state, a second state, and a third state.

FIG. 5 is a schematic structural diagram illustrating an exemplary needlestick prevention device in a first state according to some embodiments of the present disclosure.

The first state refers to a state of a needlestick prevention device after assembly and before injection actuation. In some embodiments, as illustrated in FIG. 5, in the first state, an accumulation member may be accumulated, and the fixing sleeve 20 may be in a first position. The first position refers to a position of the fixing sleeve 20 relative to the limiting sleeve 30 before injection actuation. When the fixing sleeve 20 is in the first position, the limiting buckle 34 may be connected with the locking clips 21, the at least one support plate 23 may be in the at least one second limiting hole 33, and the limiting plate 24 may be at one end of the at least one guiding feature 31 near the at least one second limiting hole 33.

In some embodiments, during an assembly phase, the fixing sleeve 20 may be pressed downward along the direction of the central axis until the locking clips 21 snap-fit into the limiting buckle 34 of the limiting sleeve 30 with assistance of a fixture. The accumulation member may be in an accumulated state when being stretched or compressed, applying a resilience force to separate the fixing sleeve 20 from the limiting sleeve 30. A relative movement of the fixing sleeve 20 and the limiting sleeve 30 may be limited by the connection between the limiting buckle 34 and the locking clips 21. An assembled needlestick prevention device may be in the first state.

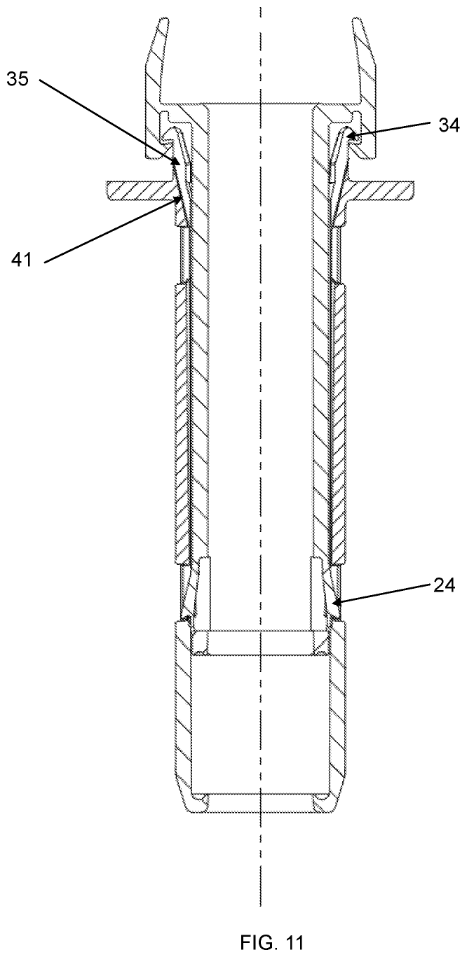

More descriptions regarding the accumulation member may be found in FIGS. 11-13 and related descriptions thereof.

In some embodiments, the limiting buckle 34 may be removably connected with the locking clip 21 through various structures. For example, the limiting buckle 34 may be snap-fit with the locking clips 21 through a buckle structure.

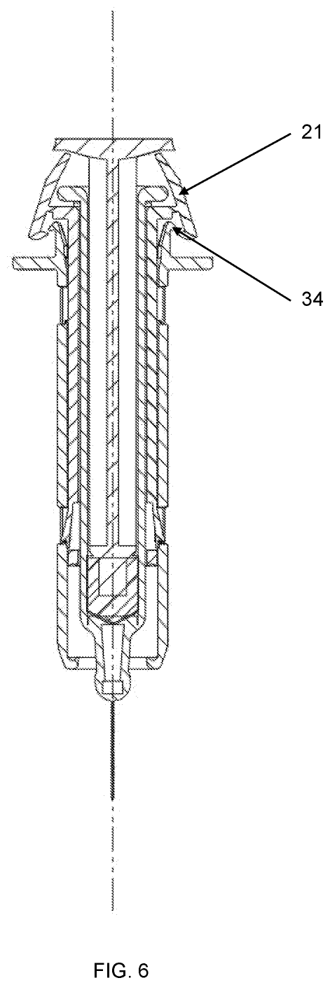

FIG. 6 is a schematic structural diagram illustrating an exemplary needlestick prevention device in a second state according to some embodiments of the present disclosure.

The second state refers to a state of a needlestick prevention device after an injection is completed. In some embodiments, as illustrated in FIG. 6, in the second state, the locking clips 21 to rotate to separate the locking clips 21 from the limiting buckle 34, the fixing sleeve 20 may be released from the limiting sleeve 30, and the accumulation member may be released.

In some embodiments, the locking clips 21 may rotate in various ways.

For example, after the injection is completed, one end of each of the locking clips 21 may be manually pressed or toggled to rotate to separate the locking clips 21 from the limiting buckle 34, and the fixing sleeve 20 may be released from the limiting sleeve 30. In this case, the accumulation member may be released. Under the action of the accumulation member, the fixing sleeve 20 may be driven to rebound, causing the fixing sleeve 20 and the limiting sleeve 30 to move away from each other along a central axis.

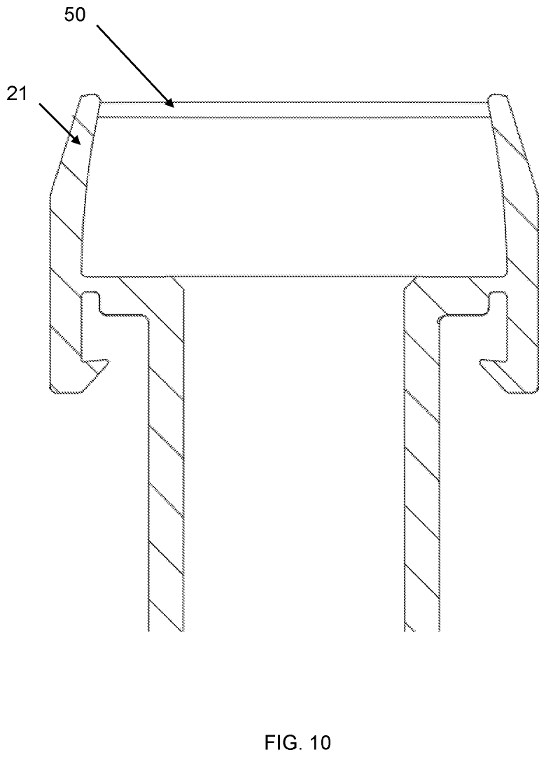

As another example, two ends of each of the locking clips 21 may be provided with a connecting strip, respectively. When the injection is completed, a pressing cap may press downward on the connecting strip to drive the locking clips to rotate by bending of the connecting strip. More descriptions may be found in FIG. 10 and related descriptions thereof.

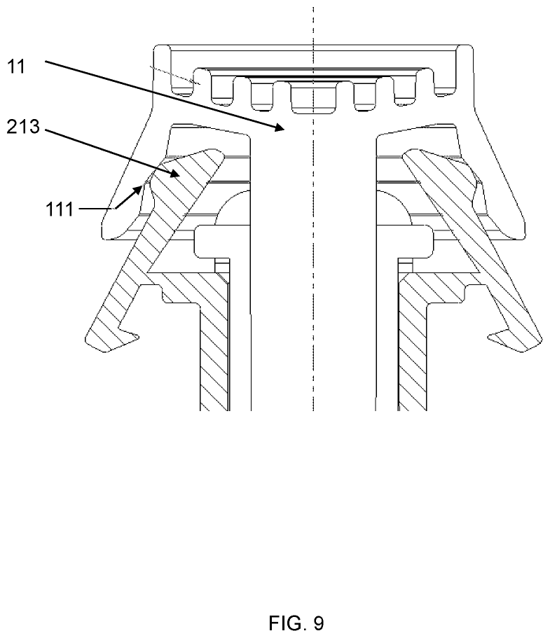

As another example, a step feature may be provided on an inner side of the pressing cap 11, and the pressing cap may be pressed downward to drive the locking clips to rotate. More descriptions may be found in FIG. 9 and related descriptions thereof.

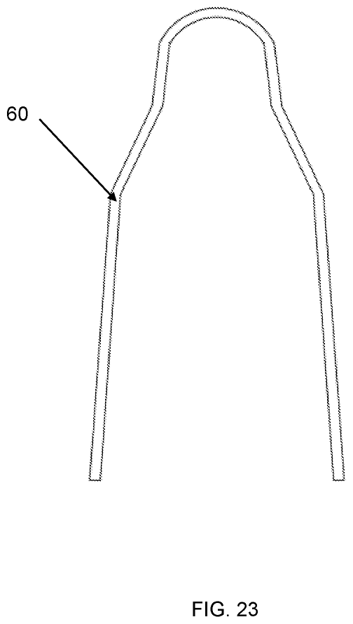

As another example, a release member may be inserted between the locking clips 21 and the limiting buckle 34. The release member may be realized to drive the locking clips to rotate. More descriptions may be found in FIG. 23 and related descriptions thereof.

A rotation manner of the locking clips 21 as described above is provided as an example only. In some embodiments, the locking clips may also rotate in other feasible ways.

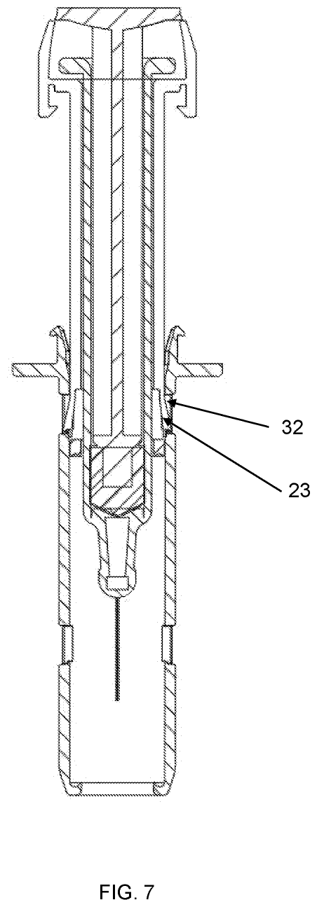

FIG. 7 is a schematic structural diagram illustrating an exemplary needlestick prevention device in a third state according to some embodiments of the present disclosure.

The third state refers to a state that an accumulation member of a needlestick prevention device is completely released. In some embodiments, as illustrated in FIG. 7, in the third state, the fixing sleeve 20 and the injector component 10 may move to a second position along the at least one guiding feature 31. The second position refers to a position of the fixing sleeve 20 relative to the limiting sleeve 30 after the accumulation member is completely released, at which the accumulation member is completely released. When the fixing sleeve 20 is in the second position, the at least one support plate 23 may be in the at least one first limiting hole 32, and the limiting plate 24 may be at one end of the at least one guiding feature 31 near the at least one first limiting hole 32.

From the second state to the third state, the accumulation member may be released to drive the fixing sleeve 20 to rebound, the at least one support plate 23 on the fixing sleeve 20 may contract inward, the limiting plate 24 may move along the at least one guiding feature, and the at least one support plate 23 may also move synchronously. When the at least one support plate 23 moves to the at least one first limiting hole 32, under an elastic force of the at least one support plate 23, the at least one support plate 23 may unfold outward, and the at least one first limiting hole 32 may limit the at least one support plate 23 from continuing to move. Since the injector component 10 and the fixing sleeve 20 are relatively fixed, the fixing sleeve 20 may drive the injector component 10 to move to the second position together.

In some embodiments of the present disclosure, the accumulation member, the locking clips, and the limiting buckle may be provided, and the locking clips may rotate to make the fixing sleeve quickly rebound under the accumulated release of the accumulation member, causing the needle to quickly retract into the limiting sleeve, thereby reducing the potential injuries of the needle to healthcare workers. The rotation of the locking clips may be achieved by manually pressing or toggling the locking clips, which is easy to operate. With the arrangement of the at least one support plate, the at least one second limiting hole, the limiting plate, the at least one guiding feature, and other structures, the fixing sleeve may be limited to the second position after the injection is completed, limiting the position of the needle to the limiting sleeve, preventing secondary pushing, thereby preventing injury, secondary infection, or the like, to personnel caused by the needle exposing again after the injection is completed. With the arrangement of the at least one support plate, the at least one first limiting hole, the limiting plate, the at least one guiding feature, and other structures, the fixing sleeve can be positioned during installation to ensure the accuracy of installation, and deviation of the limiting hole caused by the relative rotation of the fixing sleeve and the limiting sleeve can be avoided, thereby improving the accuracy of limiting.

FIG. 8 is a schematic structural diagram illustrating an exemplary cross section of a fixing sleeve according to some embodiments of the present disclosure.

In some embodiments, each of the locking clips 21 may include a connecting feature 211, a clamping rod 212, a clamping press angle 213, and a clamping hook angle 214.

The connecting feature 211 refers to a structure for connecting the clamping rod 212 with the limiting sleeve 30. In some embodiments, as illustrated in FIG. 8, the connecting feature 211 may be located in a middle of the clamping rod 212.

The clamping rod 212 refers to a part for controlling the locking and releasing of the locking clips 21. The clamping rod 212 may rotate around the connecting feature 211. In some embodiments, the clamping rod 212 may be rotatably connected with the connecting feature 211. In some embodiments, as illustrated in FIG. 8, two ends of the clamping rod 212 may be provided with the clamping press angle 213 and the clamping hook angle 214, respectively.

The clamping press angle 213 refers to a structure that may be pressed to drive the clamping rod 212 to rotate. In some embodiments, the clamping press angle 213 may be pressed in various ways. For example, the clamping press angle 213 may be manually pressed or pressed by the pressing cap 11, to drive the clamping rod 212 to rotate.

The clamping hook angle 214 refers to a structure for engaging the locking clips 21 with the limiting buckle 34.

In some embodiments, when the needlestick prevention device is in a first state, the clamping press angle 213 may be away from a central axis of the fixing sleeve 20, the clamping hook angle 214 may be near the central axis, and the clamping hook angle 214 may hook the limiting buckle 34.

In some embodiments, when the needlestick prevention device is in a second state, the clamping rod 212 may rotate around the connecting feature 211, the clamping press angle 213 may be near the central axis, the clamping hook angle 214 may be away from the central axis, and the clamping hook angle 214 may be separated from the limiting buckle 34.

In some embodiments, the connecting feature 211 may include a connecting member and a rotating shaft. The connecting member may be configured to connect the locking clips 21 to an outer side of the fixing sleeve 20. The clamping rod 212 may rotate around the rotating shaft.

FIG. 9 is a schematic structural diagram illustrating an exemplary cross section of a pressing cap according to some embodiments of the present disclosure.

In some embodiments, as illustrated in FIG. 4, a diameter of the pressing cap 11 may gradually increase along a direction from away from the fixing sleeve 20 to near the fixing sleeve 20. A step feature 111 may be provided on an inner side of the pressing cap 11. A diameter of the step feature 111 may gradually increase along a direction away from the fixing sleeve 20 to near the fixing sleeve 20.

In some embodiments, as illustrated in FIG. 9, when the pressing cap 11 is pressed downward, the clamping press angle 213 of the locking clips 21 may be compressed into the pressing cap 11. During a downward pressing process, the diameter of the step feature 111 contacted with the clamping press angle 213 may gradually decrease, causing the clamping press angle 213 to rotate in a direction close to a central axis, thereby causing the locking clips 21 to rotate. After rotation, the clamping press angle 213 may be near the central axis, and the clamping hook angle 214 may be away from the central axis.

In some embodiments, the step feature may be provided on the pressing cap, so that the locking clips and the limiting buckle can be separated after the injection is completed without manual separation. After the pressing cap is released, an elastic force of an accumulation member may automatically drive the fixing sleeve to move upward, thereby improving the operating efficiency, and preventing personnel injuries or secondary infection caused by needle exposure due to the fact that the healthcare worker forgets to perform the separation operation.

FIG. 10 is a schematic diagram illustrating an exemplary connecting strip according to some embodiments of the present disclosure.

In some embodiments, a needlestick prevention device may further include a connecting strip 50. The connecting strip 50 may be configured to connect at least two locking clips 21.

The connecting strip 50 may be configured to connect a plurality of locking clips 21. In some embodiments, the connecting strip 50 may be a rigid rope, such as a wire rope or a fiber cord.

In some embodiments, the connecting strip 50 may drive the locking clips 21 to rotate.

In some embodiments, at least two locking clips 21 may be provided. Merely by way of example, as illustrated in FIG. 10, the connecting strip 50 may be fixedly connect to upper ends of the two locking clips 21. When an injection is completed, the pressing cap 11 may be pressed downward to bend the connecting strip 50, shortening a distance between the two locking clips 21, thereby pulling the two locking clips 21 to rotate.

In some embodiments, the connecting strip may connect the at least two locking clips, the at least two locking clips may rotate after the injection is completed, and the fixing sleeve may be separated from the limiting sleeve, so that a needle can be quickly retracted without manual rotation of the at least two locking clips, thereby improving operating efficiency, and preventing personnel injuries and secondary infection caused by needle exposure.

In some embodiments of the present disclosure, each of the at least two locking clips may include a connecting feature, a clamping rod, a clamping press angle, and a clamping hook angle, thereby making the at least two locking clips more accurately realize rotation and limiting functions. With arrangement of the clamping press angle and the clamping hook angle, the at least two locking clips may realize rotation in various ways, such as manual rotation and automatic rotation, thereby meeting the needs in different scenarios.

In some embodiments, as illustrated in FIG. 4, the limiting buckle 34 may include a hook angle feature 341. The hook angle feature 341 refers to a hook structure provided at an upper end of the limiting buckle 34. The limiting buckle 34 may hook the at least two locking clips 21 through the hook angle feature 341. In some embodiments, the hook angle feature 341 may match the clamping hook angle 214 of the at least two locking clips 21.

In some embodiments, if the needlestick prevention device in a first state, the hook angle feature 341 may be snap-fit with the clamping hook angle 214 to limit the fixing sleeve 20 to a first position.

In some embodiments, the fixing sleeve may be fixed to the limiting sleeve by matching the hook angle feature with the clamping hook angle.

In some embodiments, an angle between the hook angle feature 341 and the clamping hook angle 214 may be within a range of 75°-88°.

Merely by way of example, as illustrated in FIG. 8, an angle α corresponding to the clamping hook angle 214 may be within a range of 75°-88°. For example, the angle α may be set to 75°, 80°, 88°, or the like. Merely by way of example, as illustrated in FIG. 4, an angle β corresponding to the hook angle feature 341 may be within a range of 75°-88°. For example, the angle β may be set to 75°, 80°, 88°, or the like. In some embodiments, the angle α of the clamping hook angle 214 may be the same as the angle β of the hook angle feature 341, so that the hook angle feature 341 and the clamping hook angle 214 may be more tightly snap-fit. For example, the angle α and angle β may be both set to 80°.

In some embodiments, by setting the angle of the hook angle feature and the clamping hook angle to be within the range of 75°-88°, respectively, the problem that the limiting buckle and the at least two locking clips are hooked too tightly and not easy to be disengaged due to the fact that the hook angle is too small can be solved, and the problem that the limiting buckle and the at least two locking clips are easy to loosen due to the fact that the hook angle is too large can also be solved.

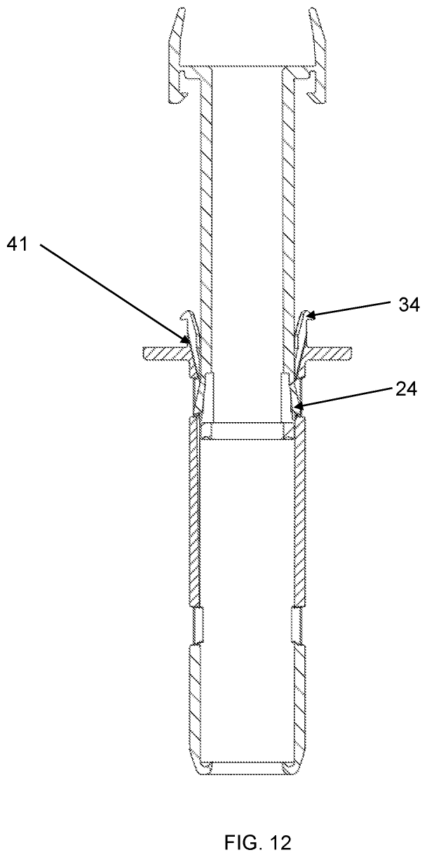

FIG. 11 is a schematic structural diagram illustrating an exemplary cross section of an accumulation member when the accumulation member is accumulated according to some embodiments of the present disclosure. FIG. 12 is a schematic structural diagram illustrating an exemplary cross section of an accumulation member when the accumulation member is released according to some embodiments of the present disclosure.

In some embodiments, as illustrated in FIG. 11, the accumulation member may be an elastic rope 41. The elastic rope refers to a rope that has elasticity. For example, the elastic rope may be a rubber band, or the like. One end of the elastic rope 41 may be fixed to the limiting buckle 34 of the limiting sleeve 30, and another end of the elastic rope 41 may be fixed to the limiting plate 24 of a fixing sleeve 20.

In a first state, illustrated in FIG. 11, the elastic rope 41 may be in a taut state. The elastic rope 41 may be fixed to the limiting buckle 34 and the limiting plate 24 in an assembly phase, and an installation length may be adjusted, so that the elastic rope 41 is taut.

In a second state, the fixing sleeve 20 may be released from the limiting sleeve 30, an elastic force of the elastic rope 41 may be released, and the elastic rope 41 may retract.

In the second state, the fixing sleeve 20 and the injector component 10 may be driven to move by the elastic rope 41. In a third state, as illustrated in FIG. 12, the fixing sleeve 20 may move to a second position along the at least one guiding feature 31. In this case, the elastic force of the elastic rope 41 may be completely released, a distance between the limiting plate 24 and the limiting buckle 34 may decrease, and the elastic rope 41 may be in a slack state.

In some embodiments, as illustrated in FIG. 11, the limiting sleeve may be provided with a rope passage groove, and the elastic rope 41 may pass through the rope passage groove 35. In some embodiments, the elastic rope 41 is movable in the rope passage groove 35.

In some embodiments, by providing the elastic rope, convenient release of the injector component can be achieved by the elastic release of the elastic rope.

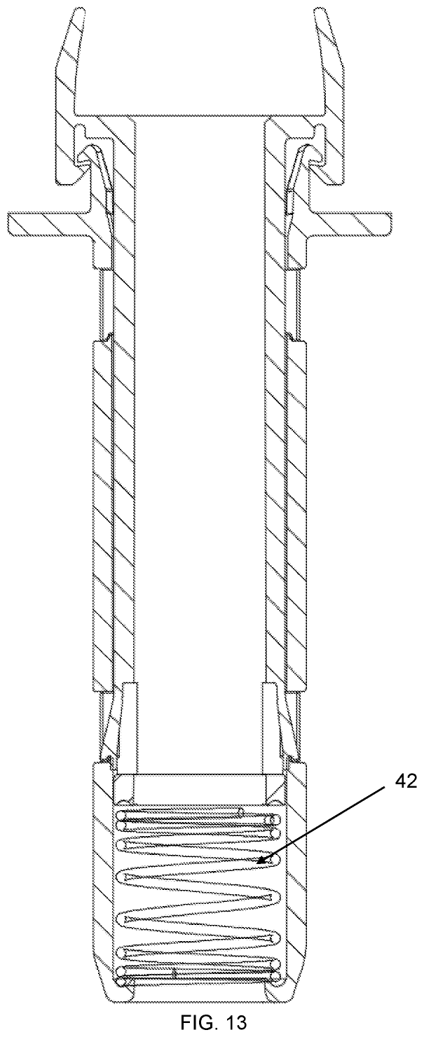

FIG. 13 is a schematic structural diagram illustrating an exemplary cross section of an accumulation member when the accumulation member is a spring according to some embodiments of the present disclosure.

In some embodiments, the accumulation member may be a spring 42. The spring 42 may be provided between the fixing sleeve 20 and the limiting sleeve 30. One end of the fixing sleeve 20 near the spring 42 and one end of the limiting sleeve 30 near the spring may be provided with a curved mounting groove matching the spring 42, respectively. In an assembly phase, the spring may be mounted between the fixing sleeve 20 and the limiting sleeve 30 through the curved mounting groove.

In a first state, the spring 42 may compressed in the curved mounting groove to store energy.

In a second state, the fixing sleeve 20 may be released from the limiting sleeve 30, and the spring 42 may rebound to push the fixing sleeve 20 and the injector component 10 to move.

In a third state, the fixing sleeve 20 may move to a second position along the at least one guiding feature 31. When the fixing sleeve 20 is in the second position, an accumulated force of the spring 42 may be completely released, and the spring 42 may be in a slack state.

By setting the spring, an elastic release of the spring may drive the fixing sleeve and the injector component to move, thereby making the release of the injector component faster and easier.

In some embodiments, a diameter of coils at two ends of the spring 42 may be larger than a diameter of a coil in a middle of the spring 42, which prevents the spring 42 from touching an inner wall of the limiting sleeve 30 when the spring 42 rebounds, and reduces friction to enable the spring 42 to quickly rebound open.

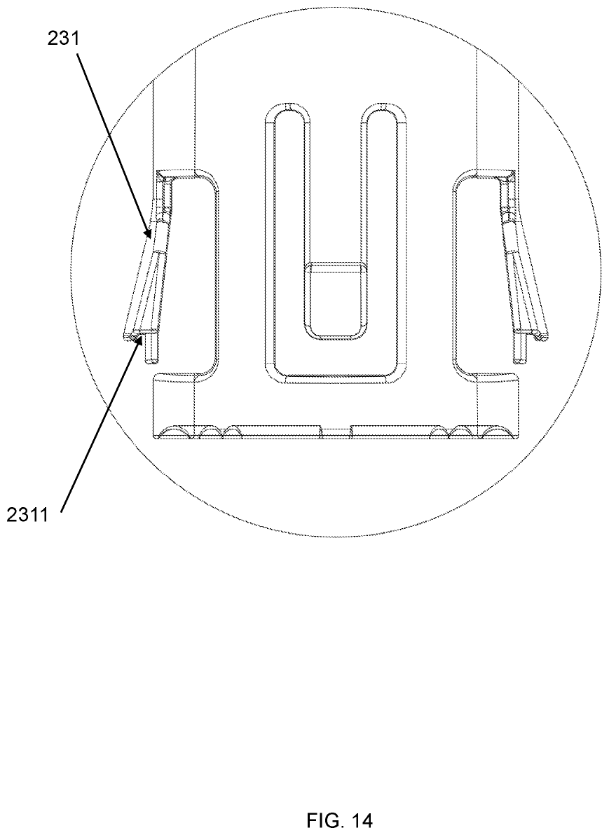

FIG. 14 is schematic diagram illustrating an exemplary local structure of a fixing sleeve according to some embodiments of the present disclosure.

In some embodiments, the at least one support plate 23 may include a bending portion 231 and a reinforcing groove 2311 provided on the bending portion.

The reinforcing groove 2311 refers to a groove structure provided on the bending portion 231. Merely by way of example, as illustrated in FIG. 14, the reinforcing groove 2311 may be provided at a lower end of the bending portion 231. In some embodiments, the reinforcing groove 2311 may make the at least one support plate Y-shaped.

In some embodiments, the limiting sleeve 30 may further include an accommodating cavity. The accommodating cavity refers to a cavity structure in the limiting sleeve 30. The accommodating cavity may be configured to accommodate the fixing sleeve 20. The accommodating cavity and the fixing sleeve 20 may be coaxially provided.

In some embodiments, a maximum distance between the bending portion 231 and a central axis of the fixing sleeve 20 may be greater than an inner diameter of the accommodating cavity, so that the bending portion 231 may achieve a limiting effect on the fixing sleeve 20.

In some embodiments, by providing the bending portion and the reinforcing groove on the at least one support plate, the limiting effect and the supporting effect of the at least one support plate can be strengthened.

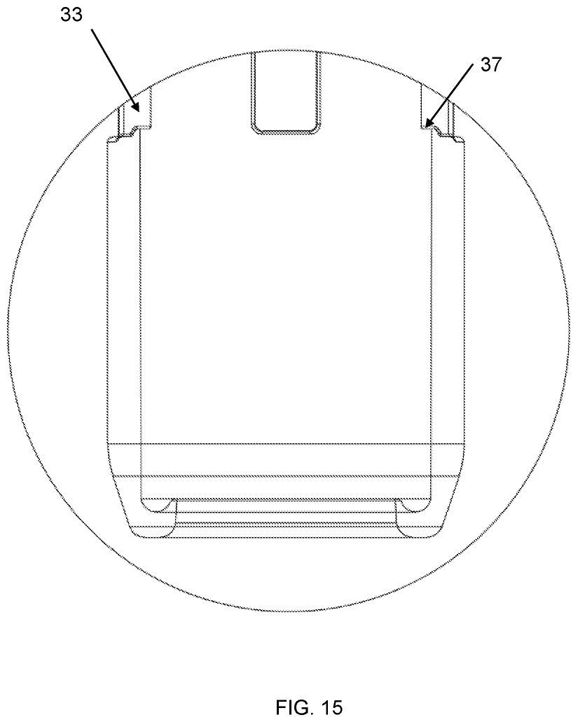

FIG. 15 is schematic diagram illustrating an exemplary local structure of a limiting sleeve according to some embodiments of the present disclosure.

In some embodiments, the at least one first limiting hole 32 and the at least one second limiting hole 33 may be provided with a reinforcing protrusion, respectively. The reinforcing protrusion refers to a protruding structure provided on the at least one first limiting hole 32 and the at least one second limiting hole 33, respectively. For example, as illustrated in FIG. 15, a first reinforcing protrusion 37 may be provided at a bottom of the at least one second limiting hole 33. In some embodiments, the reinforcing protrusion may match the reinforcing groove 2311, thereby realizing a supporting and limiting effect on the fixing sleeve 20 and the limiting sleeve 30.

In some embodiments, the supporting and limiting effect can be more significant by matching the reinforcing protrusion with the reinforcing groove.

In some embodiments, two support plates 23, two first limiting holes 32, and two second limiting holes 33 may be provided, respectively.

In some embodiments, the two support plates 23 may be provided in various ways. For example, the two support plates 23 may be symmetrically disposed on an outer side of the fixing sleeve 20 with an axis of symmetry as a central axis. Positions of the two first limiting holes 32 and the two second limiting holes 33 may be provided corresponding to the two support plates 23, respectively along a direction of the central axis. The two first limiting holes 32 may be provided at an upper end of the limiting sleeve 30. The two second limiting holes 33 may be provided at a lower end of the limiting sleeve 30.

In some embodiments, the limiting and supporting effect of the limiting sleeve on the fixing sleeve can be strengthened by providing the two support plates, the two first limiting holes, and the two second limiting holes.

FIG. 16 is a schematic diagram illustrating an exemplary local structure of at least one support plate and a limiting plate according to some embodiments of the present disclosure.

In some embodiments, the limiting plate 24 may be provided on the at least one support plate 23, and the limiting plate 24 may be a convex corner. In some embodiments, the limiting plate 24 and the at least one support plate 23 may be provided as an integrated unit, as illustrated in FIG. 16.

In some embodiments, the at least one guiding feature 31 may be a groove provided between the at least one first limiting hole 32 and the at least one second limiting hole 33. A dimension of the convex corner of the limiting plate 24 may match a dimension of the groove of the at least one guiding feature 31. The convex corner of the limiting plate 24 may slide in the groove of the at least one guiding feature 31 along the direction of the central axis.

In some embodiments, a length of the groove may be greater than a travel of the at least one support plate 23 moving from the at least one second limiting hole 33 to the at least one first limiting hole 32.

In some embodiments, a depth of the groove may be less than a thickness of the limiting sleeve 30, thereby ensuring a limiting effect of the at least one guiding feature 31.

In some embodiments, a width of the groove may be less than a width of the at least one first limiting hole 32, thereby ensuring a limiting effect of the at least one first limiting hole 32.

In some embodiments, the depth of the groove may be less than the thickness of the limiting sleeve 30, and the width of the groove may be less than the width of the at least one first limiting hole 32, thereby ensuring the limiting effect of the at least one guiding feature 31 and the at least one first limiting hole 32.

In some embodiments, the travel of the at least one guiding feature can be synchronized with that of the limiting hole by setting the limiting plate and the at least one support plate as an integrated unit. Meanwhile, a structure of the fixing sleeve can be simplified, which makes an assembly and a use of a needlestick prevention device more convenient, and reduces production cost. The setting of the depth and the width of the groove ensures that at least one of the limiting hole and the at least one guiding feature can realize the lifting effect.

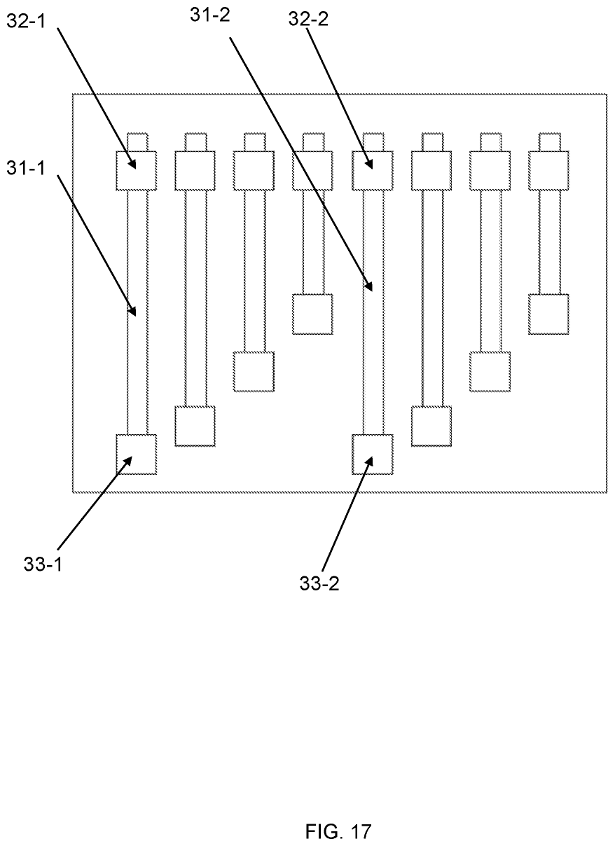

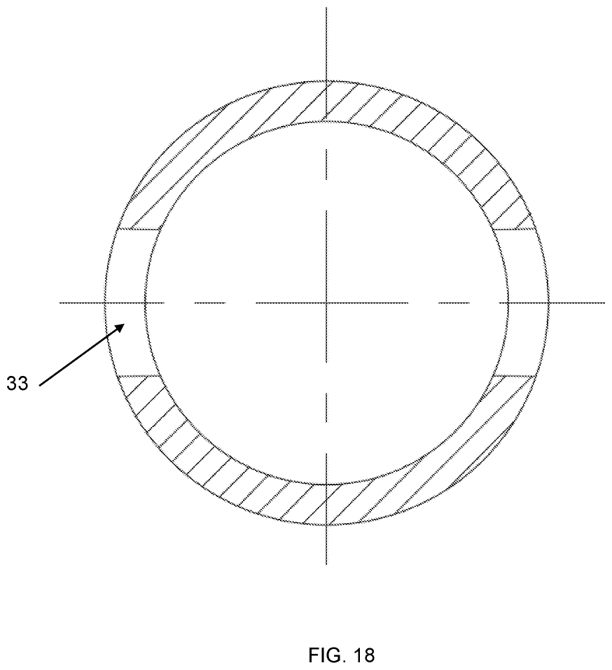

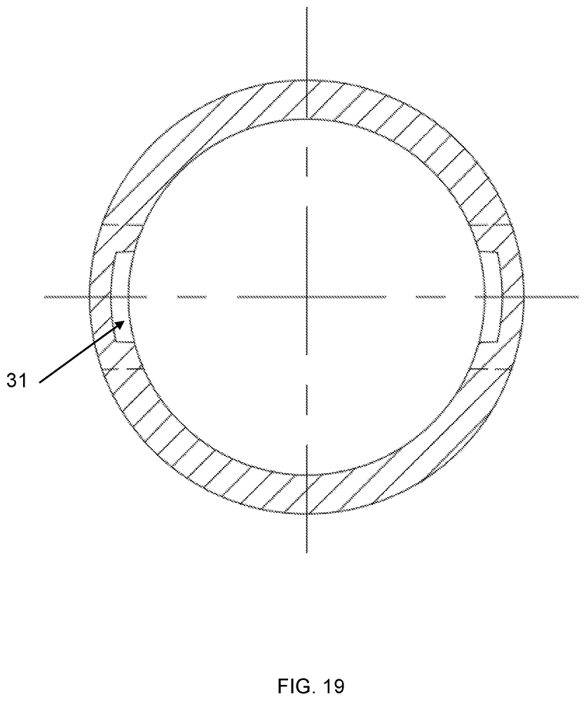

FIG. 17 is a schematic diagram illustrating an exemplary unfolding of an inner wall structure of a limiting sleeve according to some embodiments of the present disclosure. FIG. 18 is a top view illustrating an exemplary second limiting hole according to some embodiments of the present disclosure. FIG. 19 is a top view illustrating an exemplary guiding feature according to some embodiments of the present disclosure.

In some embodiments, at least two first limiting holes 32, at least two second limiting holes 33, and at least two guiding features 31 may be provided, respectively. Lengths of the at least two guiding features 31 may be different. At least one second limiting hole 33 corresponding to a longest guiding feature of the at least two guiding features 31 may be provided near a bottom of the limiting sleeve 30.

It should be understood that different lengths of the guiding features 31 may be applied to different sizes of needles 16. In an assembly phase, the limiting plate 24 and the at least one support plate 23 may be set to correspond to different lengths of the guiding features based on different lengths of needles, thereby enhancing the adaptability of a needlestick prevention device to different needles.

In some embodiments, a plurality of sets of first limiting holes 32, second limiting holes 33, and guiding features 31 may be provided. Each set of the plurality of sets of first limiting holes 32, second limiting holes 33, and guiding features 31 may include at least two first limiting holes 32, at least two second limiting holes 33, and at least two guiding features 31. For example, as illustrated in FIG. 17, four sets of first limiting holes 32, second limiting holes 33, and guiding features 31 may be provided. A first set of the four sets of first limiting holes 32, second limiting holes 33, and guiding features 31 may include a first limiting hole 32-1, a first limiting hole 32-2, a second limiting hole 33-1, a second limiting hole 33-2, a guiding feature 31-1, and a guiding feature 31-2. Similarly, a second set of the four sets of first limiting holes 32, second limiting holes 33, and guiding features 31 may include two first limiting holes, two second limiting holes, and two guiding features. A third set of the four sets of first limiting holes 32, second limiting holes 33, and guiding features 31 may include two first limiting holes, two second limiting holes, and two guiding features. A fourth set of the four sets of first limiting holes 32, second limiting holes 33, and guiding features 31 may include two first limiting holes, two second limiting holes, and two guiding features. Since the four sets corresponding to four lengths of the guiding features, four travels of the at least one support plate moving from the second limiting holes to the first limiting holes may also be provided. The four travels may be applied to four different lengths of needles.

In some embodiments, at least two first limiting holes 32, at least two second limiting holes 33, and at least two guiding features 31 may be provided, respectively. For example, as illustrated in FIG. 18 and FIG. 19, lengths of the two guiding features 31 may be different. One first limiting hole, one second limiting hole, and one guiding feature may correspond to one size of needle, thereby meeting he use of two different sizes of needles.

In some embodiments of the present disclosure, a needlestick prevention device can be applied to different lengths of needles by providing a plurality of travels between the first limiting holes and the second limiting holes. The second limiting holes can be provided close to the bottom to increase the travels between the first limiting holes and the second limiting holes, making the needlestick prevention device adapt to a longer needle, thereby increasing application scenarios, and meeting the diverse needs of users.

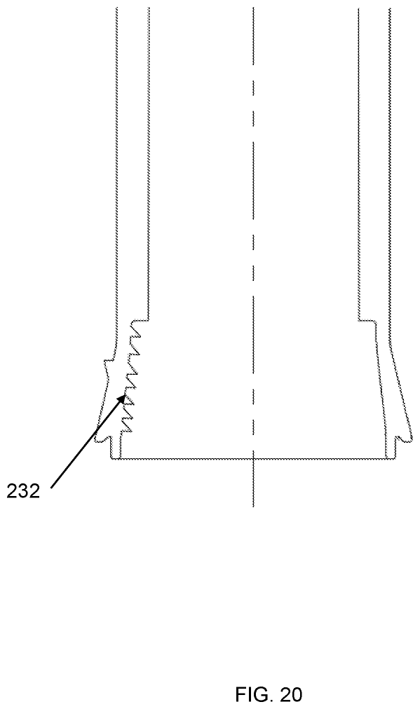

FIG. 20 is a schematic diagram illustrating an exemplary adjustment portion according to some embodiments of the present disclosure.

In some embodiments, the at least one support plate 23 may be provided with an adjustment portion 232 of a predetermined shape. The adjustment portion may include at least one fixing position. An accumulation member is capable of mating with the at least one fixing position.

The adjustment portion 232 refers to a structure for adjusting the accumulation member. For example, the adjustment portion 232 may be configured to adjust an elasticity of the accumulation member.

The fixing position refers to a position on the adjustment portion 232 for fixing the accumulation member. For example, different fixing positions may correspond to different elasticities of the accumulation member.

The predetermined shape refers to a pre-set shape for adjusting the adjustment portion 232. The predetermined shape may include various shapes. For example, the predetermined shape may be a serrated shape, a hooked shape, a looped shape, or the like.

Merely by way of example, as illustrated in FIG. 20, the adjustment portion 232 may be the serrated shape. Different serrated positions may be different fixing positions of the adjustment portion 232.

In some embodiments, the accumulation member may be the elastic rope 41. The elastic rope 41 may be fixed at different serrated positions to correspond to different lengths at which the elastic rope 41 is elongated. By adjusting the serrated position at which the elastic rope 41 is fixed, the length at which the elastic rope 41 is elongated may be adjusted, thereby adjusting the elasticity of the elastic rope 41. It should be understood that the closer the serrated position is to a bottom of the at least one support plate 23, the longer the length of the elastic rope 41 is elongated, the greater the elasticity, and the longer matching travel and the at least one guiding feature 31 may be selected.

In some embodiments, the elasticity of the accumulation member can be adjusted by setting the predetermined shape of the adjustment portion, so that the elasticity of the accumulation member can better match different guiding features, thereby avoiding the problem that a limiting effect of the fixing sleeve is weakened or even absent caused by excessive or insufficient elasticity leading to mismatch between a retracting travel and the at least one guiding feature.

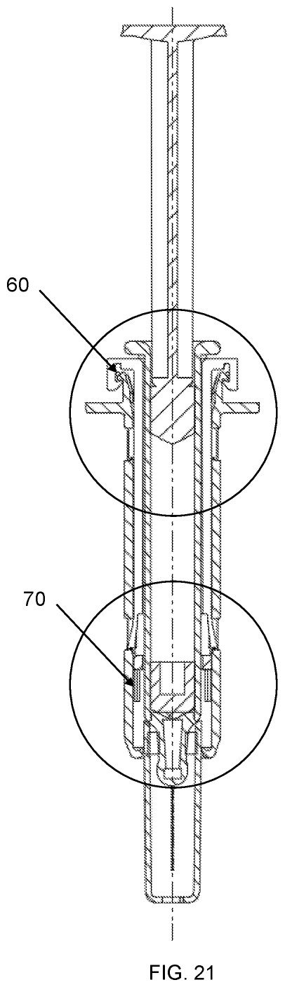

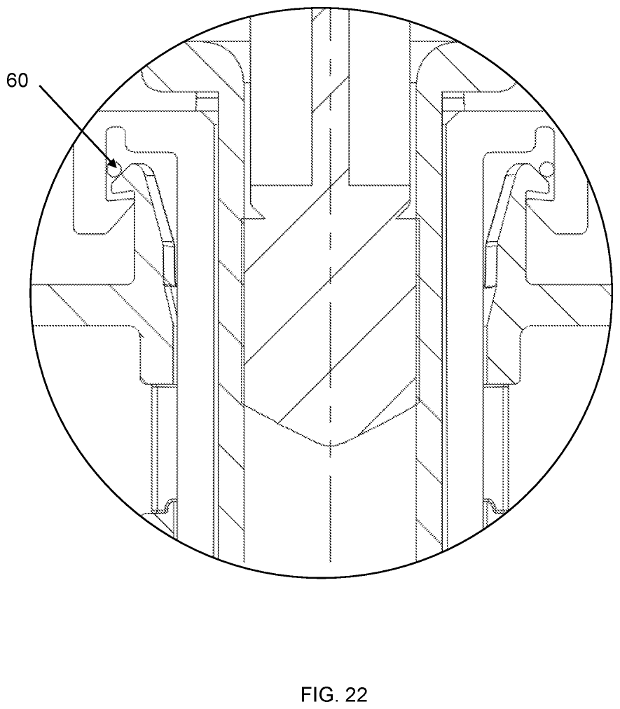

FIG. 21 is a schematic structural diagram illustrating an exemplary needlestick prevention device in a first state according to some embodiments of the present disclosure. FIG. 22 is a schematic diagram illustrating an exemplary local enlargement of a needlestick prevention device in a first state according to some embodiments of the present disclosure. FIG. 23 is a schematic diagram illustrating an exemplary release member of a needlestick prevention device according to some embodiments of the present disclosure.

In some embodiments, as illustrated in FIG. 21, the needlestick prevention device may further include a release member 60. In some embodiments, the locking clips 21 may include the clamping hook angle 214. The release member 60 is capable of being inserted between the locking clips 21 and the limiting buckle 34. Merely by way of example, as illustrated in FIG. 22, the release member 60 may be inserted into a gap between the locking clips 21 and the limiting buckle 34.

The release member 60 refers to a part for separating the clamping hook angle 214 from the limiting buckle 34. In some embodiments, the release member 60 may have an elasticity. In some embodiments, as illustrated in FIG. 23, the release member 60 may be a U-shaped snap pin.

In some embodiments, the release member 60 may be released to drive the locking clips 21 to rotate. For example, the release member 60 may be manually inserted between the locking clips 21 and the limiting buckle 34, and may be released by manual pulling or pressing. Merely by way of example, if the release member 60 is the U-shaped snap pin and inserted, and the release member 60 may be accumulated and extend under the elasticity the release member 60, thereby providing a limiting effect. After being pressed or pulled out, an accumulated release of the release member 60 may drive the locking clips 21 to rotate.

In some embodiments, by setting the release member to drive the locking clips to rotate, the locking clips only needs to be provided with a clamping hook angle, and may not need to be provided with a clamping press angle, which simplifies structures of the locking clips, and reduces the difficulty of manufacturing, thereby reducing the production cost. Meanwhile, the release member has a simple structure, is easy to manufacture, and has a low cost, which can further reduce the cost.

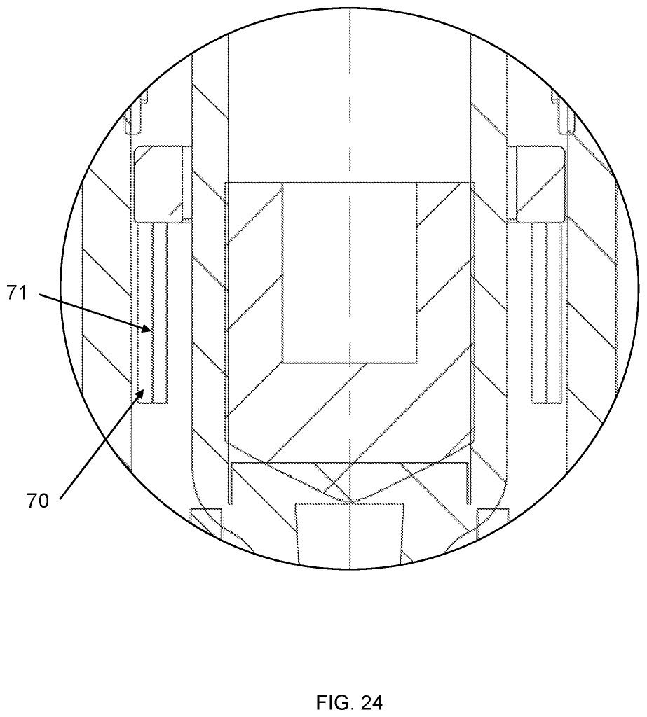

FIG. 24 is a schematic diagram illustrating an exemplary local structure of a needlestick prevention device according to some embodiments of the present disclosure.

In some embodiments, the needlestick prevention device may further include a cooling member.

A cooling member 70 refers to a part that has a heat-absorbing cooling function. In some embodiments, the cooling member 70 may be provided with an isolating layer 71. The isolating layer 71 may be configured to separate the cooling member into two inner chambers. Each of the two inner chambers may be provided a coolant. The coolant refers to a chemical agent that undergoes a heat-absorbing reaction. The two inner chambers of the cooling member 70 may be each filled with a different cooling chemical reagent, such as water and ammonium chloride or ammonium nitrate, or another inorganic salt, or barium hydroxide octahydrate and ammonium chloride.

In some embodiments, the cooling member 70 may be arranged at one end of the fixing sleeve 20 near the needle 16 and connected with the fixing sleeve 20.

The isolating layer 71 refers to a part for separating the cooling member 70. In some embodiments, the isolating layer 71 may be a thin and low-toughness material, such as thin rigid plastic or polycarbonate. An outer side of the cooling member 70 may be made of an elastic material, and a toughness of the outer side of the cooling member 70 may be much greater than that of the isolating layer. When the cooling member 70 is subjected to an external force, the isolating layer 71 may be easily damaged, while the outer surface of the cooling member 70 may not be easily damaged.

In some embodiments, after an injection is completed, the isolating layer 71 may be destroyed by kneading or extruding, allowing the chemical reagents in different inner chambers to come into contact, resulting in a heat-absorbing reaction that lowers a local temperature of a space where a needle is located, thereby reducing a temperature of the needle.

After the injection is completed, liquid may remain on the needle, such as a patient's bodily fluid, medicinal fluid, etc. Local cooling of the space where the needle is located through the cooling member may slows down a spreading movement of the fluid on the needle, which further reduces the risk of bacterial infection.

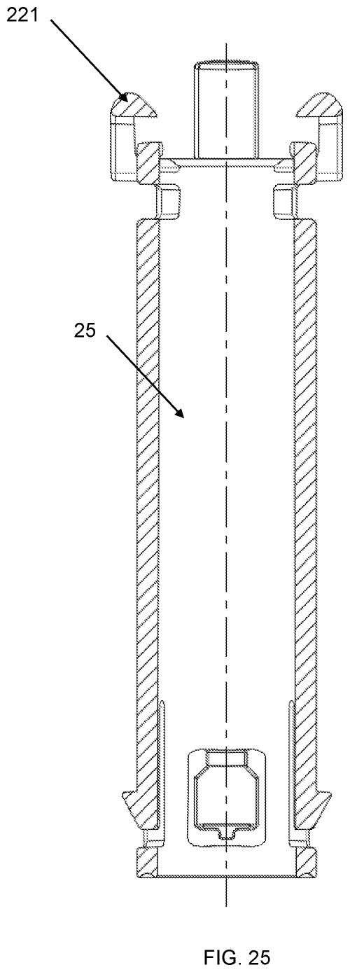

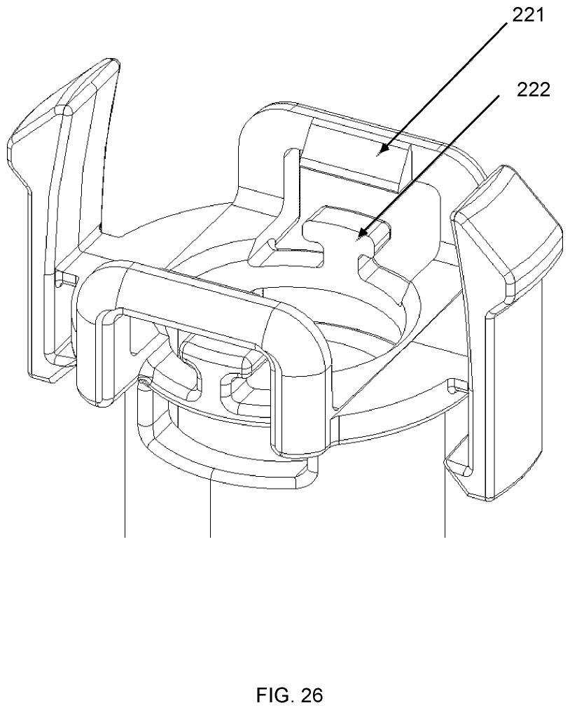

FIG. 25 is a cross-sectional diagram illustrating an exemplary fixing protrusion and a fixing cavity according to some embodiments of the present disclosure. FIG. 26 is a stereoscopic diagram illustrating an exemplary fixing protrusion and a fixing cavity according to some embodiments of the present disclosure.

In some embodiments, as illustrated in FIG. 26, the flanging fixing clip 22 may include fixing protrusions 221 and deforming members 222. In some embodiments, two fixing protrusions 221 and two deforming members 222 may be provided, respectively, thereby providing a better fixing effect. The two fixing protrusions 221 may be symmetrically disposed at an outer side of the flanging fixing clip 22 with respect to a central axis. Positions of the two deforming members 222 may be located below the two fixing protrusions 221.

In some embodiments, both the two fixing protrusions 221 and the two deforming members 222 may be made of an elastically deformable material.

Each of the two fixing protrusions 221 refers to a protruding structure on the flanging fixing clip 22. As illustrated in FIG. 26, each of the two fixing protrusions 221 may be provided at an upper end of the flanging fixing clip 22. The injector syringe flanging 14 may be snap-fit between the two fixing protrusions 221 and the two deforming members 222 to be fixed on the fixing sleeve 20.

Each of the two deforming members 222 refers to a part on the flanging fixing clip 22 for deforming. In some embodiments, each of the two deforming members 222 may be provided in various shapes, such as an elongated strip shape or a T shape. Each of the two deforming members 222 illustrated in FIG. 26 may be T-shaped. In some embodiments, a groove may be provided at a position of each of the two deforming members 222 near the fixing cavity 25, thereby providing a deformation space for the deforming member 222.

In some embodiments, the fixing sleeve may further include the fixing cavity 25.

The fixing cavity 25 refers to a hollow structure inside the fixing sleeve 20 for accommodating the injector syringe 15. In some embodiments, the injector syringe 15 may be disposed in the fixing cavity 25, and the injector syringe flanging 14 may be disposed outside the fixing cavity 25. For example, as illustrated in FIG. 25, the fixing cavity 25 and the fixing sleeve 20 may be coaxially provided.

In some embodiments, a minimum distance between the two fixing protrusions 221 may be less than a minimum outer diameter of the injector syringe flanging 14, so that the two fixing protrusions 221 may limit the injector syringe flanging 14 from moving upward.

In some embodiments, an inner diameter of each of the two deforming members 222 may be equal to an inner diameter of the fixing cavity 25, and a vertical distance between each of the two deforming members 222 and each of the two fixing protrusions 221 may be less than a thickness of the injector syringe flanging 14, so as to ensure that when the injector syringe flanging 14 is snap-fit between each of the two fixing protrusions 221 and each of the two deforming members 222, each of the two fixing protrusions 221 may abut against a lower end of the injector syringe 15 and an upper end of each of the two deforming members.

In some embodiments of the present disclosure, by providing the two fixing protrusions and the two deforming members, the injector syringe flanging can be securely fixed in the flanging fixing clip of the fixing cartridge, thereby avoiding secondary injuries caused by a movement of a needle driven by the injector syringe.

In some embodiments, an elastic modulus of each of the two deforming members 222 may be less than an elastic modulus of each of the two fixing protrusions 221.

It should be understood that the lower the elastic modulus, the relatively larger the elastic modulus deformation, the smaller the stiffness, the easier the material is to deform, and the better the flexibility. The higher the elastic modulus, the relatively smaller the elastic modulus deformation that occurs in a material, the greater the stiffness, the less likely the material is to deform, the more brittle the material is. Each of the two deforming members has a relatively low elastic modulus, which makes each of the two deforming members flexible, and beneficial for securely fixing a larger injector syringe flanging to the flanging fixing clip. Each of the two fixing protrusions has a relatively high elastic modulus and great stiffness, which is beneficial for limiting downward sliding of the injector syringe, thereby improving the applicability of the flanging fixing clip, and enhancing the fixing effect.

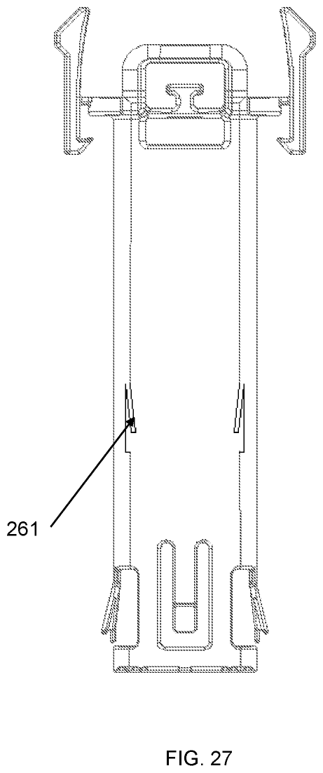

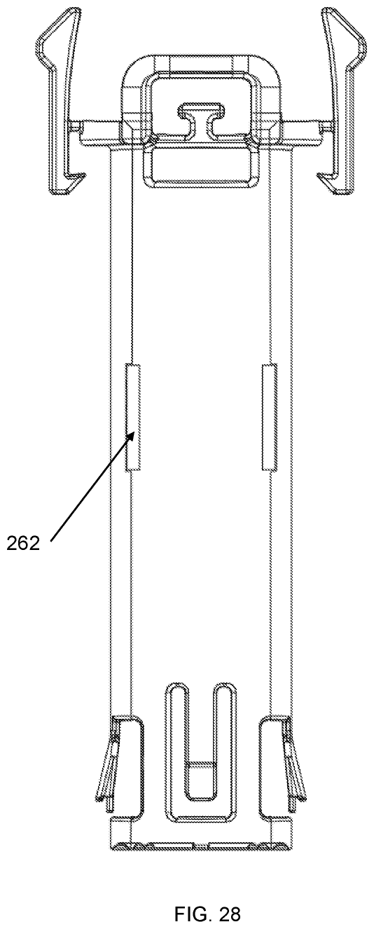

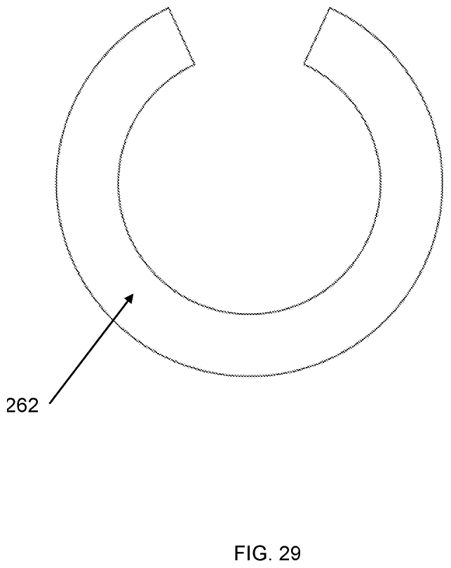

FIG. 27 is a schematic diagram illustrating an exemplary position of an adjustment member according to some embodiments of the present disclosure. FIG. 28 is a schematic diagram illustrating an exemplary position of an adjustment member according to some embodiments of the present disclosure. FIG. 29 is a schematic diagram illustrating an exemplary adjustment member according to some embodiments of the present disclosure.

In some embodiments, an adjustment member may be provided at an inner side of the fixing sleeve 20. The adjustment member refers to a protruding structure for fixing the injector syringe 15. In some embodiments, the adjustment member may be located at the inner side of the fixing sleeve 20.

In some embodiments, the adjustment member may be configured to adapt to different diameters of the injector syringes 15. The adjustment member 26 may be an elastic structure, such that an elasticity of the adjustment member may change when different diameters of the injector syringes 15 are pressed downward, thereby adapting to different diameters of the injector syringes 15.

In some embodiments, as illustrated in FIG. 26, the adjustment member may include a bending edge 261. The bending edge 261 may be arranged near the needle 16. As illustrated in FIG. 27, the bending edge 261 may bend towards a central axis of the fixing sleeve 20. Merely by way of example, the adjustment member may be structurally similar to the at least one support plate 23, except that the bending edge 261 of the adjustment member may bend inwards, while the at least one support plate 23 may bend outwards.

When different diameters of the injector syringes 15 are pressed downward, the bending edges 261 of the adjustment member may be subjected to different degrees of compression and bending, so that different diameters of the injector syringes may be fixed under a rebound force of the bending edge 261.

In some embodiments, as illustrated in FIG. 29, the adjustment member may be a ring-shaped open elastic hoop 262. In some embodiments, as illustrated in FIG. 28, the ring-shaped open elastic hoop 262 may elastically hoop a middle of an inner side of the fixing sleeve 20.

When different diameters of the injector syringes 15 are pressed downward, an opening of the ring-shaped open elastic hoop 262 may be expanded or narrowed, thereby fixing different diameters of the injector syringes 15. For example, a structure of the ring-shaped open elastic hoop 262 may be illustrated in FIG. 29.

The ring-shaped open elastic hoop 262 may elastically hoop the inner side of the fixing sleeve 20, so that different diameters of the injector syringes 15 may be elastically fixed without affecting an existing structure. In some embodiments, a plurality of ring-shaped open elastic hoops of different sizes may also be provided for the product to meet different needs.

In some embodiments, the inner side of the fixing sleeve 20 may be provided with a groove matching a thickness of the ring-shaped open elastic hoop 262. The ring-shaped open elastic hoop 262 may be fixedly disposed in a middle of the fixing sleeve 20 through the groove. In an assembly phase, when a diameter of the injector syringe 15 is small, the ring-shaped open elastic hoop 262 may sleeve an outer side of the injector syringe 15. The ring-shaped open elastic hoop 262 may be pressed downward with an installation process of the injector syringe 15 until the ring-shaped open elastic hoop 262 touches the groove to be blocked. Then the ring-shaped open elastic hoop 262 may be fixed in the groove.

In some embodiments, by providing the adjustment member, the plurality of ring-shaped open elastic hoops of different sizes may be fixed to the inner side of the fixing sleeve by the elastic force of the adjustment member, thereby enabling the needlestick prevention device to be applicable to different diameters of injector syringes, and increasing the application scenarios.

The basic concept has been described above. Obviously, for those skilled in the art, the above detailed disclosure is only an example, and does not constitute a limitation to the present disclosure. Although not expressly stated here, those skilled in the art may make various modifications, improvements and corrections to the present disclosure. Such modifications, improvements and corrections are suggested in this disclosure, so such modifications, improvements and corrections still belong to the spirit and scope of the exemplary embodiments of the present disclosure.

Meanwhile, the present disclosure uses specific words to describe the embodiments of the present disclosure. For example, “one embodiment”, “an embodiment”, and/or “some embodiments” refer to a certain feature, structure or characteristic related to at least one embodiment of the present disclosure. Therefore, it should be emphasized and noted that references to “one embodiment” or “an embodiment” or “an alternative embodiment” two or more times in different places in the present disclosure do not necessarily refer to the same embodiment. In addition, certain features, structures, or characteristics in one or more embodiments of the present disclosure may be properly combined.

In addition, unless clearly stated in the claims, the sequence of processing elements and sequences described in the present disclosure, the use of counts and letters, or the use of other names are not used to limit the sequence of processes and methods in the present disclosure. While the foregoing disclosure has discussed by way of various examples some embodiments of the invention that are presently believed to be useful, it should be understood that such detail is for illustrative purposes only and that the appended claims are not limited to the disclosed embodiments, but rather, the claims are intended to cover all modifications and equivalent combinations that fall within the spirit and scope of the embodiments of the present disclosure. For example, although the implementation of various components described above may be embodied in a hardware device, it may also be implemented as a software only solution, e.g., an installation on an existing server or mobile device.

Similarly, it should be noted that in order to simplify the presentation of the disclosure of this specification, and thereby aid in the understanding of one or more embodiments of the invention, the foregoing descriptions of embodiments of this specification sometimes group multiple features together in a single embodiment, accompanying drawings, or in a description thereof. However, this method of disclosure does not imply that more features are required for the objects of the present specification than are mentioned in the claims. Rather, claimed subject matter may lie in less than all features of a single foregoing disclosed embodiment.

In some embodiments, counts describing the quantity of components and attributes are used. It should be understood that such counts used in the description of the embodiments use the modifiers “about”, “approximately” or “substantially” in some examples. Unless otherwise stated, “about”, “approximately” or “substantially” indicates that the stated figure allows for a variation of ±20%. Accordingly, in some embodiments, the numerical parameters used in the disclosure and claims are approximations that can vary depending upon the desired characteristics of individual embodiments. In some embodiments, numerical parameters should consider the specified significant digits and adopt the general digit retention method. Although the numerical ranges and parameters used in some embodiments of the present disclosure to confirm the breadth of the range are approximations, in specific embodiments, such numerical values are set as precisely as practicable.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

What is claimed is:

1. A needlestick prevention device, comprising an injector component, an accumulation member, a fixing sleeve, and a limiting sleeve; wherein the fixing sleeve and the accumulation member are arranged in the limiting sleeve; one end of the accumulation member is fixedly connected with the fixing sleeve, and another end of the accumulation member is fixedly connected with the limiting sleeve;
the injector component includes a pressing cap, an injector syringe flanging, an injector syringe, and a needle;
the fixing sleeve includes one or more locking clips, a flanging fixing clip, at least one support plate, and a limiting plate, and the flange fixing clip is configured to fix the injector syringe flanging;
the limiting sleeve includes at least one guiding feature, at least one first limiting hole, at least one second limiting hole, and a limiting buckle;
the needlestick prevention device has a first state, a second state, and a third state; wherein
in the first state, the accumulation member is accumulated, and the fixing sleeve is in a first position; when the fixing sleeve is in the first position, the limiting buckle is connected with the locking clips, the at least one support plate is in the at least one second limiting hole, and the limiting plate is at one end of the at least one guiding feature near the at least one second limiting hole;
in the second state, the locking clips rotate to separate from the limiting buckle, the fixing sleeve is released from the limiting sleeve, and the accumulation member is released; and
in the third state, the fixing sleeve and the injector component move along the at least one guiding feature to a second position; and when the fixing sleeve is in the second position, the support plate is in the at least one first limiting hole, and the limiting plate is at one end of the at least one guiding feature near the at least one first limiting hole.

2. The needlestick prevention device of claim 1, wherein each of the locking clips includes a connecting feature, a clamping rod, a clamping press angle, and a clamping hook angle; and the clamping rod is capable of rotating around the connecting feature;
the connecting feature is arranged in a middle of the clamping rod, and two ends of the clamping rod are the clamping press angle and clamping hook angle, respectively;
in the first state, the clamping press angle is away from a central axis of the fixing sleeve, and the clamping hook angle is close to the central axis; and
in the second state, the clamping press angle is near the central axis, and the clamping hook angle is away from the central axis.

3. The needlestick prevention device of claim 2, wherein the limiting buckle includes a hook angle feature, and the hook angle feature matches the clamping hook angle; and
in the first state, the hook angle feature is engaged with the clamping hook angle to limit the fixing sleeve to the first position.

4. The needlestick prevention device of claim 3, wherein an angle of the hook angle feature and the clamping hook angle is within a range of 75°-88°, respectively.

5. The needlestick prevention device of claim 1, wherein the accumulation member is an elastic rope, one end of the elastic rope is fixed to the limiting buckle of the limiting sleeve, and another end of the elastic rope is fixed to the limiting plate of the fixing sleeve.

6. The needlestick prevention device of claim 5, wherein the limiting sleeve is provided with a rope passage groove, and the elastic rope passes through the rope passage groove.

7. The needlestick prevention device of claim 1, wherein the at least one support plate includes a bending portion and a reinforcing groove arranged on the bending portion; the limiting sleeve further includes an accommodating cavity configured to accommodate the fixing sleeve; and
a maximum distance between the bending portion and a central axis of the fixing sleeve is greater than an inner diameter of the accommodating cavity.

8. The needlestick prevention device of claim 7, wherein the at least one first limiting hole and the at least one second limiting hole are provided with a reinforcing protrusion, respectively, and the reinforcing protrusion matches the reinforcing groove.

9. The needlestick prevention device of claim 7, further comprising two support plates, two first limiting holes, and two second limiting holes.

10. The needlestick prevention device of claim 1, wherein the limiting plate is arranged on the at least one support plate, the limiting plate is a convex corner, the at least one guiding feature is a groove arranged between the at least one first limiting hole and the at least one second limiting hole; and
a depth of the groove is less than a thickness of the limiting sleeve, or a width of the groove is less than a width of the first limiting hole.

11. The needlestick prevention device of claim 10, wherein at least two sets of first limiting holes, second limiting holes, and guiding features are provided, respectively, lengths of each set of the guiding features are different, and each set of the at least two sets of first limiting holes, second limiting holes, and guiding features include at least two first limiting holes, at least two second limiting holes, and at least two guiding features; and in the at least two second limiting holes, a second limiting hole corresponding to a longest guiding feature of the at least two guiding features is arranged near a bottom of the limiting sleeve.

12. The needlestick prevention device of claim 11, wherein the support plate is provided with an adjustment portion with a predetermined shape, the adjustment portion includes at least one fixing position; and the accumulation member is capable of mating with the at least one fixing position.

13. The needlestick prevention device of claim 1, further comprising a connecting strip configured to connect with at least two locking clips.

14. The needlestick prevention device of claim 1, further comprising a release member, wherein each of the locking clips includes a clamping hook angle, and the release member is capable of being inserted between the locking clips and the limiting buckle.

15. The needlestick prevention device of claim 1, wherein the flanging fixing clip includes two fixing protrusions and two deforming members;
the fixing sleeve further includes a fixing cavity, the injector syringe is arranged in the fixing cavity, and the injector syringe flanging is arranged outside the fixing cavity; and
a minimum distance between the two fixing protrusions is less than a minimum outer diameter of the injector syringe flanging.

16. The needlestick prevention device of claim 15, wherein an elastic modulus of each of the two deforming members is less than an elastic modulus of each of the two fixing protrusions.

17. The needlestick prevention device of claim 1, further comprising a cooling member, wherein the cooling member is provided with an isolating layer configured to separate the cooling member into two inner chambers; and
the cooling member is arranged at one end of the fixing sleeve near the needle and connected with the fixing sleeve.

18. The needlestick prevention device of claim 1, wherein an inner side of the fixing sleeve is provided with an adjustment member configured to adapt to different diameters of injector syringes;
the adjustment member includes a bending edge which is arranged near the needle and bends towards a central axis of the fixing sleeve; or
the adjustment member is a ring-shaped open elastic hoop which tightly encircles a middle of the inner side of the fixing sleeve.

19. The needlestick prevention device of claim 1, wherein the injector component includes a plunger, the plunger includes a rubber plug and a push plug which are detachably fixed, and a pre-filled medication is sealed between the push plug and the needle.

20. The needlestick prevention device of claim 19, wherein the injector component further includes a needle cap.
